(19) United States
(12) Patent Application Publication
(10) Pub. No.: US 2006/0064011 A1
(43) Pub. Date: Mar. 23, 2006

(54) Ventricular injection catheter

(76) Inventor: Mun Kyung Hong, New York, NY (US)

Correspondence Address: Browdy & Neimark, PLLC;Suite 300, 624 Ninth Street, N.W., Washington, DC, 20001-5303, US

(21) Appl. No.: 11/231,793

(22) Filed: Sep. 22, 2005

Related U.S. Application Data
(60) Provisional application No. 60/612,553, filed on Sep. 23, 2004.

(51) Int. Cl. A61M 25/00

(52) U.S. Cl. 600/435; 604/264

(57) ABSTRACT

A catheter for delivery of contrast material for a medical imaging procedure, the catheter comprising: (a) an operative distal head comprising a tubular body including at least two turns and characterized by a transverse proximal aspect that is larger than a transverse distal aspect; and (b) a plurality of contrast material ports distributed along said tubular body between at least two of said at least two turns.

The present application claims the benefit under 35 USC 119(e) of U.S. Provisional Application No. 60/612,553 filed on Sep. 23, 2004; entitled “NOVEL LEFT VENTRICULOGRAPHY CATHETER” the disclosure of which is incorporated herein by reference.

### FIELD OF THE INVENTION

The present invention relates to a catheter for delivery of contrast material, for example, to the left ventricle.

### BACKGROUND OF THE INVENTION

Catheterization of the heart is an established medical technique. Catheters may be used to deploy medical devices (e.g. stents), for medical intervention (e.g. balloon angioplasty), or to deliver contrast material to a target to facilitate imaging.

One procedure which relies upon contrast material delivery is left ventriculography. The accuracy of assessment of left ventricular function, valvular abnormalities, and regional wall motion abnormality is correlated to contrast material delivery throughout the ventricle.

Currently available catheters for ventriculography may be divided into two categories, “pigtail” catheters and multipurpose catheters. Inherent shortcomings of these available designs reduce the quality of ventriculography data. Both designs tend to induce ventricular arrhythmias when used for ventriculography, by a mechanism of mechanical irritation of the ventricle.

Pigtail catheters are characterized by a planar loop at the distal end of the catheter which resembles a pig's tail. The large “pigtail” distal end of the catheter which is typically inserted toward the left ventricle apex often induces ventricular arrhythmia which leads to disintegration of the left ventriculogram.

Similarly, the curved distal tip of a multipurpose catheter also frequently points upward and contacts the myocardium once inside the left ventricle. This causes ventricular arrhythmia during systole. These arrhythmias lead to disintegration of the left ventriculogram.

Some mechanical irritation is caused by the pigtail or multipurpose catheters being frequently “trapped” behind the papillary muscle, causing further arrhythmia. Another source of mechanical irritation is the rigidity of the catheters, including the fixed angulation of the distal tip in the left ventricle and the shaft immediately outside the left ventricle. Currently available catheters typically do not flex to accommodate contractions of the left ventricle in the area of the ventricular apex and/or portion of the catheter immediately outside the ventricle (e.g. in the aorta).

In order to avoid arrhythmia, the catheter may be positioned in the ventricular base. This can cause artificial mitral regurgitation of injected contrast material and/or premature ejection from the left ventricle during ventriculography under high pressure. This premature ejection prevents acquisition of meaningful data despite delivery of a volume of contrast material. This requires a repeat of the insertion and delivery of contrast material, unnecessarily exposing the patient to an additional volume of contrast material.

Alternatively or additionally, positioning the catheter in the ventricular base may lead to inadequate opacification of the apex due to insufficient or uneven delivery of contrast material.

Another problem peculiar to the pigtail catheter is that its “pigtail” loop is difficult to insert into the arterial sheath. This may cause the catheter tip to become kinked or cause jets of blood to spray onto the operator.

Both the pigtail and multipurpose catheters are typically disconnected from the pressure monitoring system and reconnected to a power injector for injection of a large volume of contrast material under high pressure to opacify the left ventricle or aorta. This practice can increase the procedure time which may increase the probability of formation of blood clots. In many cases, concern over this issue leads to administration of blood thinning medication to a patient before a procedure. Alternatively or additionally, this practice can introduce air into the injection system and result in complications such as injury or death.

The following references are offered as being indicative of previously available catheter configurations of the pigtail and multipurpose type and contrast material delivery apparatus. The list does not purport to be exhaustive.

U.S. Pat. No. 5,480,392, issued to Mous, teaches a curved cardiac catheter which delivers contrast material from a plurality of ports along its length as well as from a distal tip. Mous specifies that the distal end has a “permanent curvature”.

U.S. Pat. No. 5,876,386, issued to Samson, teaches a cardiac catheter with controllable stiffness and a distal tip with a helical configuration.

U.S. Pat. No. 6,701,180 and U.S. Pat. No. 5,857,464, issued to Desai, teach a flexible endocardial catheter which employs axial slits for contrast material delivery. Contraction of the distal portion of the catheter causes the slits to open.

U.S. Pat. No. 5,085,635, issued to Cragg, teaches use of multiple ports near a closed distal tip for contrast material dispersion.

U.S. Pat. No. 6,361,528, issued to Wilson, teaches a flexible catheter which expands in cross section during use.

U.S. Pat. No. 5,037,403 issued to Garcia, teaches a flexible curved catheter with multiple contrast material ports.

U.S. Pat. No. 5,163,431, issued to Griep, teaches a catheter with a curved end, multiple contrast material ports and different portions with different degrees of flexibility.

U.S. Pat. No. 5,299,574, issued to Bower, teaches a catheter with a specifically configured end part to conform to an aorta wall and a coronary cusp.

U.S. Pat. No. 5,267,982, issued to Sylvanowicz, teaches that a curved configuration of a distal portion of a catheter can be varied while the catheter is in the patient.

U.S. Pat. No. 5,593,385, issued to Harrison, teaches a dispensing apparatus for contrast media.

U.S. Pat. No. 5,476,453, issued to Mehta, teaches curved catheters configured for concurrent injection of contrast material into two coronary arteries.

U.S. Pat. No. 5,489,278, issued to Abrahamson, teaches elongated contrast material ports in a catheter.

U.S. Pat. No. 4,748,984, issued to Patel teaches a catheter with multiple contrast material ports.

U.S. Pat. No. 4,694,838 to Wijayarthna teaches a coronary catheter with a helical portion transverse to the main catheter axis.

U.S. Pat. No. 4,735,620 to Ruiz and U.S. Pat. No. 4,961,731 to Bodicky teach an angiographic catheter with a curved flexible tip and contrast material ports positioned to cause mixing of contrast material via intersecting currents.

U.S. Pat. No. 4,986,814 to Burney does not relate directly to cardiac applications although it teaches a catheter with a helical portion with inward facing holes.

U.S. Pat. No. 4,531,933 to Norton does not relate directly to cardiac applications although it teaches a flexible silicone tubing with a transverse helical portion.

The specifications of all publications and/or patents cited above are fully incorporated herein by reference to the same extent as if each had been individually incorporated herein by reference.

### SUMMARY OF THE INVENTION

An aspect of some embodiments of the present invention relates to a flexible cardiac catheter with an intraventricular portion characterized by a large transverse proximal aspect. Optionally, the transverse proximal aspect of the intraventricular portion is larger than a transverse distal aspect of the intraventricular aspect. In an exemplary embodiment of the invention, employing a catheter with an increased proximal aspect reduces the chance of unwanted catheter ejection during ventriculography. In an exemplary embodiment of the invention, the transverse proximal aspect of the intraventricular portion is larger than a heart valve through which the catheter has been inserted but smaller than the ventricular base. Optionally, this prevents unwanted ejection of the catheter through the valve. The valve may optionally be an aortic or mitral valve. Optionally, this reduces mechanical stimulation of the myocardium which might lead to arrhythmia,

In an exemplary embodiment of the invention, contrast material ports are distributed axially and/or radially along at least a portion of a length of a curved portion of the catheter. Optionally, the ports are positioned to aim ejected contrast material generally towards a mid-region of a ventricle. Optionally, a distal portion of the catheter contains an additional contrast material port to deliver contrast material to a ventricular apical region. In an exemplary embodiment of the invention, the contrast material ports are numbered, sized, positioned and/or otherwise configured to reduce resistance of injected contrast material and facilitate injection using a hand operated device such as a syringe which operates at a low pressure. Optionally, the ports are configured so that the injection force at any one port is insufficient to cause damage to the myocardium by penetration of the contrast material and/or insufficient to cause mechanically-imitated arrhythmia.

In an exemplary embodiment of the invention, the catheter includes a portion characterized by an undulating curve so that the catheter is substantially flat. In an exemplary embodiment of the invention, the catheter includes a portion characterized by one or more helical turns so that the catheter is three dimensional. Optionally, the curvature conforms to ventricular geometry during diastole. In an exemplary embodiment of the invention, the catheter does not contact an inner surface of the myocardium during diastole. In an exemplary embodiment of the invention, the curved portions of the catheter are more flexible than intervening portions in order to help reduce mechanical irritation of a ventricular wall during ventricular contraction. Optionally, flexibility of the catheter permits conformation of the catheter to a reduced systolic ventricular cavity size and reduces mechanical irritation. Alternatively or additionally, a portion of the catheter outside the ventricle may include a flexible joint capable of conforming to a changing angle between the intraventricular portion of the catheter and the extra ventricular (e.g. aortic) portion of the catheter. Optionally, this feature further reduces mechanical irritation and contributes to a reduction in arrhythmia.

An aspect of some embodiments of the present invention relates to a portion of the catheter outside the ventricle bent at a pre-defined angle so that the distal portion of the catheter is correctly positioned within the ventricle.

An aspect of some embodiments of the present invention relates to a method to reduce the amount of contrast material required for cardiac ventriculography by providing a large contrast exit area along the length of a curved catheter. Optionally, the contrast exit area is positioned and/or oriented to deliver contrast material to the central region of the ventricle. Optionally, a large contrast exit area is achieved by a large number of ports and/or ports with a large cross-sectional area. In an exemplary embodiment of the invention, a large number of contrast material ports permits adequate image quality with a reduced amount of contrast material. Optionally, the large contrast exit area reduces a resistance pressure for injection and/or provides efficient distribution of contrast material and/or permits use of more flexible materials in catheter construction. In an exemplary embodiment of the invention, at least 8, optionally 10, optionally 12, optionally 16 or more contrast material ports are provided. In an exemplary embodiment of the invention, the large contrast exit area eliminates the need for connection to a mechanical contrast material pump. Optionally this reduces the procedure time and/or the risk of blood clot formation.

An aspect of some embodiments of the present invention relates a method to reduce the irritation of the ventricular wall during ventriculography. Optionally, a catheter configuration which conforms to cardiac geometry reduces the irritation. Optionally, cardiac geometry refers to ventricular geometry. In an exemplary embodiment of the invention, conformation to ventricular geometry is achieved during diastole and systole via use of a flexible catheter. Optionally, a catheter configuration with a transverse proximal aspect that is larger than a transverse distal aspect provides a desired degree of conformation to ventricular geometry. Optionally, cardiac geometry includes the aortic ventricular junction. In an exemplary embodiment of the invention, conformation to the dynamic angular configuration of this junction may be achieved by introducing at least one extraventricular flex point on the catheter. Alternatively or additionally, a fixed angle in a portion of the catheter deployed in the aorta aids in correct positioning of a portion of the catheter deployed within the aorta. In an exemplary embodiment of the invention, an envelope of the catheter may conform the ventricular envelope during diastole and/or or systole.

In an exemplary embodiment of the invention, a catheter for delivery of contrast material for a medical imaging procedure is provided. The catheter includes:
(a) an operative distal head includes a tubular body including at least two turns and characterized by a transverse proximal aspect that is larger than a transverse distal aspect; and
(b) a plurality of contrast material ports distributed along said tubular body between at least two of said at least two turns.
Optionally, the catheter is sized to fit a left cardiac ventricle of an adult.
Optionally, the turns are more flexible than an intervening portion between said turns.
Optionally, the catheter additionally includes a distal region designed and configured to extend axially relative to the catheter.
Optionally, said distal region includes at least one distal contrast material port.
Optionally, at least one of said at least one distal contrast material port is aimed axially with respect to said tubular body.
Optionally, said tubular body includes at least one flexible point capable of flexion through an angle proximal to said operative distal head.
Optionally, said tubular body includes at least one fixed angle proximal to said operative distal head.
Optionally, the distances between said turns decrease with axial progression along the catheter towards a distal end.
Optionally, said turns are at least 25% more flexible than intervening portions of the catheter.
Optionally, the turns with angles in a plurality of planes cause said operative distal head to significantly deviate from a planar configuration.
Optionally, said operative distal head includes a helical portion.
Optionally, said plurality of contrast material ports includes at least 8 ports.
Optionally, the method includes,
(a) deploying a distal catheter head characterized by a transverse proximal aspect that is larger than a transverse distal aspect within the ventricle;
(b) ejecting contrast material through at least one contrast material port on said distal catheter head; and
(c) acquiring image data.

In an exemplary embodiment of the invention, there is provided a method of imaging an aorta. The method includes;
(a) deploying a distal catheter head characterized by a transverse proximal aspect that is larger than a transverse distal aspect within the aorta;
(b) ejecting contrast material through at least one contrast material port on said distal catheter head; and
(c) acquiring image data.

In an exemplary embodiment of the invention, there is provided a method for reducing the amount of contrast material injected during a medical diagnostic procedure by at least 50% The method includes employing a catheter according to claim 1.

In an exemplary embodiment of the invention, there is provided a method of reducing mechanical irritation of a ventricular wall during a medical diagnostic procedure. The method includes employing a catheter having an operative head which responds to an applied contractile force with a low resistance to deliver contrast material for the procedure. Optionally, said catheter is sufficiently elastic to alternate between a first contracted conformation and a second extended conformation in accord with a degree of externally applied force.

In an exemplary embodiment of the invention, there is provided an intraventricular catheter for delivery of contrast material for ventriculography. The catheter includes:
(a) an operative distal head which includes a tubular body including at least two flexible turns; and
(b) a plurality of contrast material ports distributed along said tubular body between at least two of said at least two turns. Optionally, said operative distal head defines a transverse proximal aspect that is larger than a transverse distal aspect.

In an exemplary embodiment of the invention, there is provided an intraventricular catheter for delivery of contrast material for ventriculography. The catheter includes a pre-defined angle at an extraventricular portion of the catheter which directs a distal portion of the catheter to a correct position during use.

### BRIEF DESCRIPTION OF FIGURES

In the figures, identical structures, elements or parts that appear in more than one figure are generally labeled with the same numeral in all the figures in which they appear. Dimensions of components and features shown in the figures are chosen for convenience and clarity of presentation and are not necessarily shown to scale. The figures are listed below.

FIGS. 1A and 1B are schematic representations of an exemplary catheter according to the invention positioned within the left ventricle during diastole and during systole respectively;

FIGS. 2A and 2B are schematic representations contrast material injection and mixing respectively employing a catheter as depicted in FIGS. 1A and 1B;

FIGS. 3A and 3B are schematic representations of an additional exemplary catheter according to the invention positioned within the left ventricle during diastole and during systole respectively; and

FIGS. 4A and 4B are schematic representations of an additional exemplary catheter according to the invention including a flexible hinge point outside the left ventricle during diastole and during systole respectively.

### DETAILED DESCRIPTION OF EMBODIMENTS

Cardiac ventricles are characterized by a wide base which tapers to a narrow apex. During ventriculography, the catheter is typically inserted through a valve at the ventricular base. In a retrograde procedure, insertion is through an aortic valve. In a transeptal procedure, insertion is through a mitral valve.

In an exemplary embodiment of the invention, a ventriculography catheter with geometry that conforms to the natural shape of the ventricle is provided. According to some exemplary embodiments of the invention, the catheter is characterized by a proximal aspect of its intraventricular portion which is larger than a distal aspect of its intraventricular portion.

In an exemplary embodiment of the invention, like prior art catheters, a catheter according to the present invention may be delivered to the ventricle, for example, by stretching from the unconstrained configuration during insertion into the arterial sheath and advancement over a guidewire into the aortic root in a retrograde approach. Alternatively or additionally, the catheter may be sufficiently flexible that insertion into the sheath is easily accomplished. At this point, the catheter can be prolapsed into the ventricle as commonly done with known “pigtail” catheters. In an exemplary embodiment of the invention, a J-tipped guidewire is used for insertion into the ventricle. In an exemplary embodiment of the invention, the catheter may then be inserted into a left ventricle over the guidewire. Alternatively or additionally, a stylette may be employed for catheter insertion. Optionally, the stylette fits in one or more of the contrast injection ports.

Regardless of the guidewire configuration employed to deliver the catheter to the ventricle, once the catheter has been positioned inside the left ventricle and the guidewire has been removed, the intraventricular portion of the catheter assumes its unconstrained form. In an exemplary embodiment of the invention, the unconstrained intraventricular portion of the catheter does not contact the myocardium during diastole.

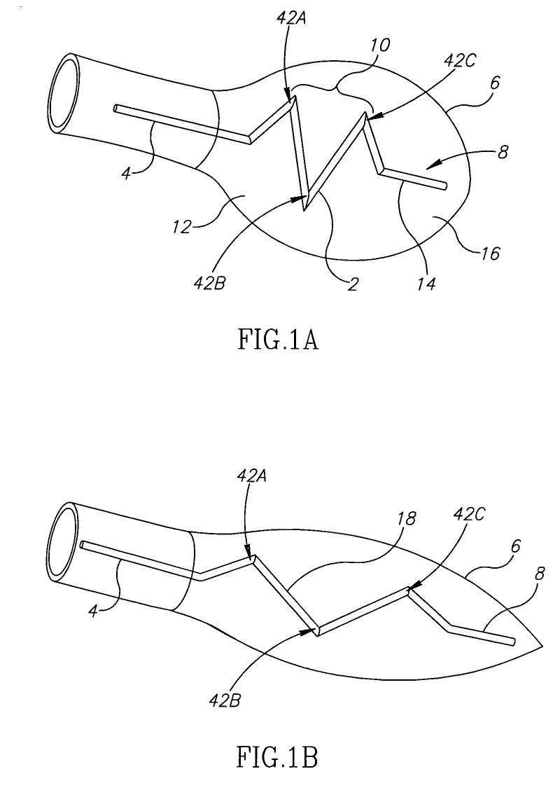

FIG. 1A illustrates an exemplary embodiment of catheter 4 according to the present invention in a sagittal section of a ventricle 6. The operative distal head will be referred to herein as intraventricular portion 2 of catheter 4. Intraventricular portion 2 of catheter 4 is deployed within ventricle 6 which is pictured in a relaxed (diastolic) state. The proximal region 10 of intraventricular portion 2 of catheter 4 remains in the ventricular base 12. Optionally, a distal tip 8 extends toward the ventricular apex 16. In an exemplary embodiment of the invention, the distal portion 14 of catheter 4 has a narrower internal diameter than proximal portion 10. Intraventricular portion 2 of catheter 4 may be described as having an undulating portion so that a distance between turn 42A and 42B is similar to a distance between turn 42B and 42C. Optionally, the undulating portion is degenerate so that a distance between turn 42A and 42B is greater than a distance between turn 42B and 42C. Optionally, the undulating portion degenerates monotonically.

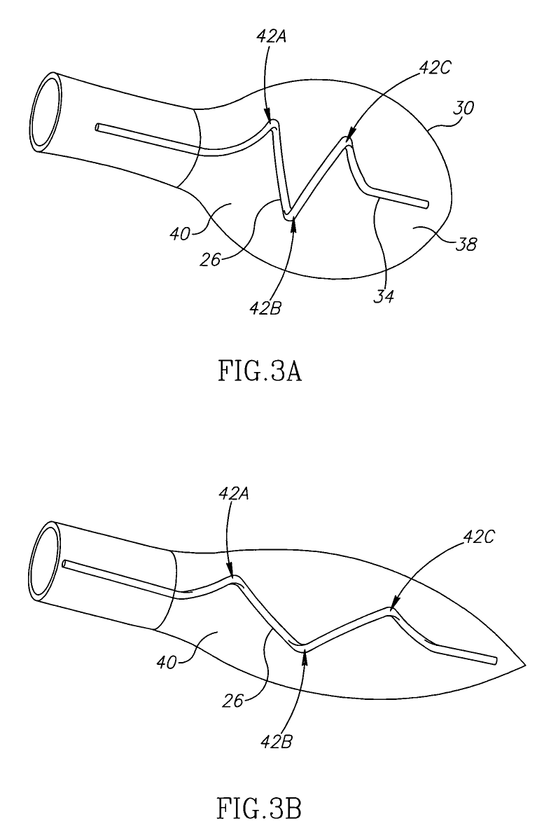

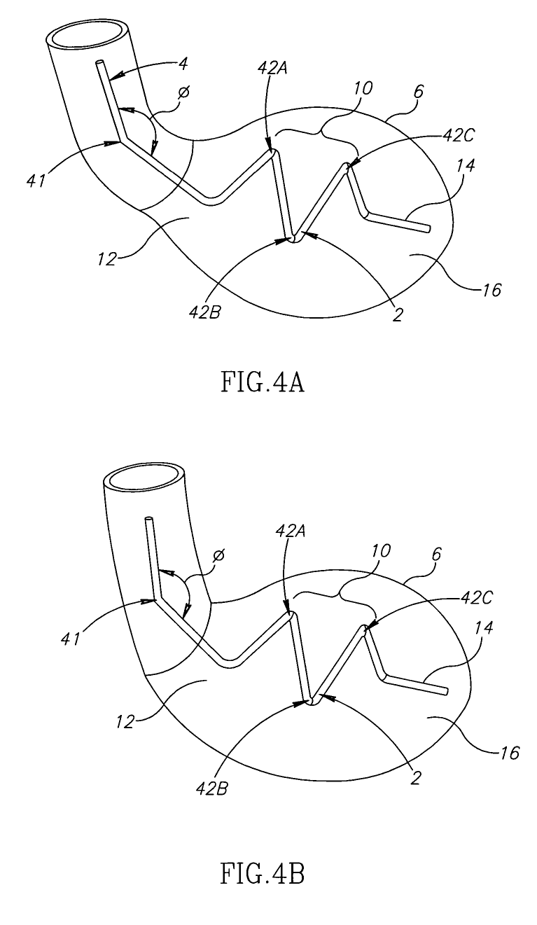

In an exemplary embodiment of the invention, proximal region 10 of intraventricular portion 2 is characterized by a transverse aspect which is smaller than a width of ventricular base 12 but larger than a transverse aspect of distal tip 8 of catheter 2. Optionally, this increased transverse aspect may be achieved by pre-forming or shaping proximal portion 10 of intraventricular portion 2 of catheter 4 so that it has a two dimensional (FIGS. 1A, 1B, 2A, 2B, 4A and 4B) and/or a three dimensional (FIGS. 3A and 3B) curve in its unconstrained state. In an exemplary embodiment of the invention, proximal portion 10 of intraventricular portion 2 describes a three dimensional curve, for example a helix.

In the FIGS. (1A, 1B, 2A and 2B) proximal portion 10 of intraventricular portion 2 is depicted as a two dimensional sigmoid curve which crosses a midline of ventricle 6 twice. In an exemplary embodiment of the invention, a catheter with three turns 42 crosses a midline of the ventricle twice. In an exemplary embodiment of the invention, a catheter with two turns crosses the midline only once. Optionally, a catheter with four or more turns in the undulating portion causes intraventricular portion 2 of catheter 4 to cross the midline three or more times.

In an exemplary embodiment of the invention; the undulating curve of proximal portion 10 of intraventricular portion 2 is a degenerate curve so that the transverse aspect of proximal portion 10 of intraventricular portion 2 decreases along catheter 4 from ventricular base 12 towards ventricular apex 16. In an exemplary embodiment of the invention, the distance from turn 42B to turn 42C is less than the distance from turn 42A to turn 42B by 10%, optionally 20%, optionally 30%, optionally 40% optionally 50% or more. A degenerate curve conforms generally to the shape of the ventricle, which is tapered from base to apex. Alternatively or additionally, a degenerate curve reduces tension in catheter construction/delivery and/or permits use of softer, more flexible, materials in catheter construction. Because the catheter shape conforms to ventricular geometry, distal tip 8 may protrude further into apex 16 to provide improved apical contrast material distribution. Optionally, delivery of contrast material into the ventricular apex is improved during diastole. In the midregion of the ventricle, a degenerate curve of proximal portion 10 of intraventricular portion 2 helps achieve diffuse contrast material delivery while reducing, optionally eliminating contact with the myocardium during diastole. In an exemplary embodiment of the invention, advancement of catheter 4 over a guidewire facilitates positioning of distal portion 14 near the apex away from the papillary muscles.

FIG. 1B shows ventricle 6 in systolic contraction with intraventricular portion 2 of catheter 4 of FIG. 1A in an altered conformation due to the changed ventricular geometry. During systole, the internal cavity of ventricle 6 is reduced in size by a rhythmic contraction from apex 16 to base 12. Size reduction of the ventricular lumen is most pronounced at apex 16. The changed ventricular geometry causes a change in the shape of proximal portion 10 of intraventricular portion 2 as a result of force from contraction of the ventricular chamber. Proximal portion 10 of intraventricular portion 2 is compressed in its transverse aspect by the contracting myocardium. This causes axial extension of proximal portion 10 of intraventricular portion 2 so that mid segment 18 is brought into closer proximity with the ventricular midline. As a result, distal portion 8 of catheter 4 is extended further into ventricular apex 16. This improves delivery of contrast material into the ventricular apex. Optionally, proximal portion 10 of intraventricular portion 2 is sufficiently soft that contact with contracting myocardium causes a degree of mechanical irritation which is insufficient to produce arrhythmia. In an exemplary embodiment of the invention, turns 42 are fashioned of a material which is more flexible than intraventricular portion 2 as a whole. Optionally, this may be achieved by reduced wall thickness. Optionally, rigid rings are embedded in a narrow catheter wall near turns 42 so that flexibility is provided while structural integrity is maintained.

In exemplary embodiments in which a three dimensional curve, such as a helical curve, is employed a similar axial extension of midsection 26 of intraventricular portion 2 occurs. (See FIGS. 3A and 3B). Optionally, a degenerate helical configuration, for example a conical helix is employed so that distal tip 34 of catheter 4 may still extend deep into ventricular apex 38 for apical delivery of contrast material.

Optionally, catheter 4 includes an additional flexible “hinge point” 41 positioned so that it is outside the ventricle (e.g. in the aorta in a retrograde procedure). Hinge point 41 permits catheter 4 to conform to changes in configuration of the aortic/ventral region via changes in flexion angle theta (φ). Optionally, this contributes to a reduction in mechanical irritation of the myocardium and/or reduces the likelihood of premature catheter ejection.

In an exemplary embodiment of the invention, once proximal portion 10 of intraventricular portion 2 is positioned within ventricle 6, its large transverse aspect prevents unwanted ejection from the ventricle and/or mitral valve regurgitation of contrast material. This is because the unconstrained conformation (FIG. 1A) provides a wider transverse aspect than the valve (e.g. aortic valve in retrograde procedure) in ventricular base 12. During systole, this transverse aspect of proximal portion 10 of intraventricular portion 2 remains sufficiently wide so that unwanted ejection of the catheter through the valve is prevented.

In an exemplary embodiment of the invention, proximal portion 10 of intraventricular portion 2 is sufficiently flexible that mechanical irritation of the myocardium is reduced. In an exemplary embodiment of the invention, flexibility of turns 42A, 42B and 42C permits conformation to the contracted ventricle and reduces mechanical irritation by reducing the resistance force against myocardial contraction. Optionally, this reduces the likelihood of ventricular arrhythmia. Optionally, reduction of ventricular arrhythmia contributes to successful completion of the ventriculography procedure and/or improved accuracy of ventriculography results. In an exemplary embodiment of the invention, the catheter is sufficiently soft so that it does not damage the myocardium on contact, but flexes in response to an applied systolic pressure. Alternatively or additionally, the degenerate curvature of the catheter causes it to extend so that those portions in contact with the myocardium slide along the myocardial surface. Optionally, turns 42A, 42B and 42C are constructed of thinner and/or more flexible materials. Optionally, use of thinner and/or more flexible materials reduces mechanical irritation. Optionally, mechanical irritation may be caused by myocardial contact with the catheter. In an exemplary embodiment of the invention, the turns are at least 15%, optionally 25%, optionally 50% or more, more flexible than adjacent portions of the catheter.

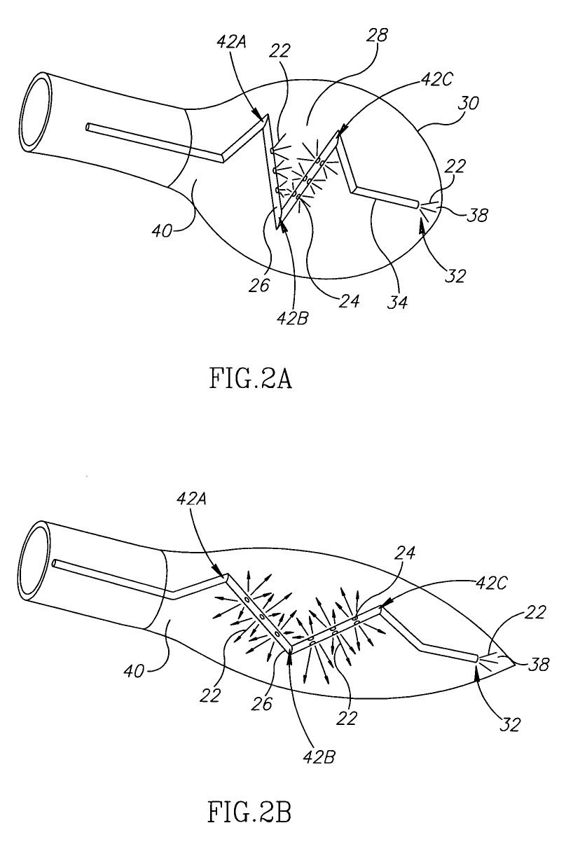

Ventriculography relies upon injection of contrast material into the ventricle. For this reason, ventriculography catheters are equipped with contrast material ports to deliver contrast material into the ventricle. FIG. 2A shows contrast material ports 24 in a catheter of the type depicted in FIG. 1A. In an exemplary embodiment of the invention, injection of contrast material is primarily into the middle of the ventricle following stabilization of the catheter position. Subsequent systolic contraction assures distribution of the contrast material throughout the ventricle by distributing ports 24 along the ventral midline as shown in FIG. 2B. Optionally, opacification of the entire ventricle is achieved. In an exemplary embodiment of the invention, opacification of the ventricle is achieved in less than 5, optionally less than 4 optionally less than three optionally 2 or fewer diastolic/systolic cycles.

FIG. 2A illustrates injection of contrast 22 through contrast material ports 24 in midsection 26 of the intraventricular portion of the catheter according to an exemplary embodiment of the invention. The contrast material is ejected primarily into the midregion 28 of the ventricle 30. Optionally, contrast material may be ejected outwards towards the myocardium. Optionally, ports 24 are positioned to take advantage of flow patterns in the ventricle. Optionally, a smaller amount of contrast material 22 is ejected from catheter distal tip 34 through an additional distal port 32. Optionally, distal port 32 faces axially towards ventricular apex 38.

FIG. 2B illustrates mixing and/or distribution of contrast material 22 during systolic contraction of ventricle 30. In an exemplary embodiment of the invention, contrast material 22 is distributed into the apex 38 and the base 40 of ventricle 30 during systole. Optionally, this improves image quality and/or reduces the amount of contrast material required by improving distribution within the ventricle and/or increasing the amount of contrast delivered in a short period of time.

In an exemplary embodiment of the invention, a large number of contrast material ports 24 are provided. Optionally the large number is between 8 and 20, optionally between 12 and 16. Optionally, each of the contrast material ports has diameter of 0.05, optionally 0.06, optionally 0.07, optionally 0.1 inches or more. Optionally, large port diameters and/or large number of ports reduce resistance in the catheter during injection. Optionally, decreased resistance in the catheter during contrast material injection reduces the Bourdon spring effect (i.e. an uncurling of curved portions) and permits more accurate delivery of contrast material to a desired region. Optionally, an applied pressure of only about 200 PSI, optionally 150 PSI, optionally 100 PSI is sufficient for contrast material injection. In an exemplary embodiment of the invention, total cross sectional area of non-distal tip contrast material ports 24 is in the range of 0.04 to 0.1, optionally 0.05 to 0.075, optionally 0.055 to 0.065, optionally about 0.0628 square inches. Optionally, a greater or smaller total cross sectional area of non-distal tip contrast material ports 24 may be employed.

Ejection of contrast material from ports 24 at low pressure provides less dispersal than ejection from similar ports at a higher pressure. In an exemplary embodiment of the invention, systolic contraction of the ventricle facilitates mixing and distribution of the contrast material in the ventricle after low pressure ejection from ports 24. Optionally, systolic mixing compensates for a smaller initial distribution of contrast material resulting from low pressure injection.

In an exemplary embodiment of the invention, a low resistance in the catheter during injection facilitates use of a hand operated contrast material delivery device (e.g. syringe). Optionally, use of a hand operated devices obviates the need for connection of an injection pump and may thus reduce the risk of creating an air embolism. Alternatively or additionally, low pressure ejection of contrast material reduces the risk of myocardial damage from a high pressure liquid stream.

In an exemplary embodiment of the invention, injection of the contrast material into mid region 28 of ventricle 30 permits adequate image quality with a reduced amount of contrast material. Optionally, savings in contrast material volume may result from efficient mixing of ejected contrast material in a subsequent systolic contraction. Optionally, injection in midregion 28 is away from apex 38 and the mitral valve. Optionally, the required amount of contrast material is reduced by 50 to 75%. Since a typical ventriculography procedure using a high pressure injection pump may require 30-35 ml of contrast material, a similar procedure according to the present invention might provide comparable image quality with less than 20 ml, optionally less than 17.5 ml, optionally less than 15 ml, optionally less than 12 ml, optionally less than 10 ml, optionally about 8 ml or less. In an exemplary embodiment of the invention, 8-12 ml of contrast material produces a satisfactory image. Optionally, reduced contrast material volume reduces patient exposure to a potentially harmful substance.

While actual physical dimensions of the catheter may vary, a catheter according to the invention constructed for use in an adult might have an intraventricular portion 2 with a length of 8 to 10 cm, height from peak to trough (e.g. 42A to 42B) of 3 to 5 cm and height from peak to trough (near ventricular apex) of 1.5 to 4 cm. Optionally, distal portion 14 might be from 0.5 to 1.5 cm, optionally about 1 cm in length. The total catheter length might be, for example, in the range of 80 to 120 cm, optionally about 100 cm. Optionally, an inside diameter of 0.01 to 0.07, optionally 0.02 to 0.06, optionally 0.03 to 0.05 optionally about 0.04 inches may be employed. An, outside diameter of 1 to 10, optionally 2 to 8, optionally 3 to 7, optionally 4-6, optionally about 5 French may be employed. Optionally, the relationship between inside diameter and outside diameter may vary along the length of intraventricular portion 2, for example to impart flexibility to turns 42A, 42B and 42C by providing a relatively thin wall at those pints. In an exemplary embodiment of the invention, the transverse proximal aspect of intraventricular portion 2 is 125 to 1755, optionally about 150% of a diameter of the aortic valve. The catheter may be constructed of plastics used in other catheter configurations, for example Pigtail catheter (Boston Scientific, Maple Grove, Minn., USA, cataloge number 16599-41). Optionally, the catheter may be sized to fit an adult ventricle and/or a child's ventricle.

After ventriculography is complete, the catheter is removed by simply drawing in backwards through the blood vessels. In an exemplary embodiment of the invention, turns 42A, 42B, and 42C and/or hinge point 41 are sufficiently flexible that they allow the catheter to straighten sufficiently for easy withdrawal through the valve, e.g. aortic valve.

Optionally, a catheter as described hereinabove may be employed in other hollows in the vasculature, such as an aorta and/or abdominal aorta and/or right ventricle. In an exemplary embodiment of the invention, the catheter is withdrawn through the aorta. Optionally, the catheter conforms to aortic geometry, optionally geometry of an aorta with an aneurysm and/or the abdominal aorta. Optionally, the catheter is paused at this point for additional contrast material ejection to facilitate aortic imaging.

In the description and claims of the present application, each of the verbs “comprise”, “include” and “have” as well as any conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to necessarily limit the scope of the invention. In particular, numerical values may be higher or lower than ranges of numbers set forth above and still be within the scope of the invention. The described embodiments comprise different features, not all of which are required in all embodiments of the invention. Some embodiments of the invention utilize only some of the features or possible combinations of the features. Variations of embodiments of the present invention that are described and embodiments of the present invention comprising different combinations of features noted in the described embodiments can be combined in all possible combinations including, but not limited to use of features described in the context of one embodiment in the context of any other embodiment. The scope of the invention is limited only by the following claims.

All publications and/or patents and/or product descriptions cited in this document are fully incorporated herein by reference to the same extent as if each had been individually incorporated herein by reference.

1. A catheter for delivery of contrast material for a medical imaging procedure, the catheter comprising:
(a) an operative distal head comprising a tubular body including at least two turns and characterized by a transverse proximal aspect that is larger than a transverse distal aspect; and
(b) a plurality of contrast material ports distributed along said tubular body between at least two of said at least two turns.

2. A catheter according to claim 1, sized to fit a left cardiac ventricle of an adult.

3. A catheter according to claim 1, wherein said turns are more flexible than an intervening portion between said turns.

4. A catheter according to claim 1, additionally comprising a distal region designed and configured to extend axially relative to the catheter.

5. A catheter according to claim 4, wherein said distal region includes at least one distal contrast material port.

6. A catheter according to claim 5, wherein at least one of said at least one distal contrast material port is aimed axially with respect to said tubular body.

7. A catheter according to claim 1, wherein said tubular body includes at least one flexible point capable of flexion through an angle proximal to said operative distal head.

8. A catheter according to claim 1, wherein said tubular body includes at least one fixed angle proximal to said operative distal head.

9. A catheter according to claim 1, wherein distances between said turns decrease with axial progression along the catheter towards a distal end.

10. A catheter according to claim 1, wherein said turns are at least 25% more flexible than intervening portions of the catheter.

11. A catheter according to claim 1, wherein turns with angles in a plurality of planes cause said operative distal head to significantly deviate from a planar configuration.

12. A catheter according to claim 11 wherein said operative distal head includes a helical portion.

13. A catheter according to claim 1, wherein said plurality of contrast material ports includes at least 8 ports.

14. A method of imaging a ventricle, the method comprising,
(a) deploying a distal catheter head characterized by a transverse proximal aspect that is larger than a transverse distal aspect within the ventricle;
(b) ejecting contrast material through at least one contrast material port on said distal catheter head; and
(c) acquiring image data.

15. A method of imaging an aorta, the method comprising,
(a) deploying a distal catheter head characterized by a transverse proximal aspect that is larger than a transverse distal aspect within the aorta;
(b) ejecting contrast material through at least one contrast material port on said distal catheter head; and
(c) acquiring image data.

16. A method for reducing for reducing the amount of contrast material injected during a medical diagnostic procedure by at least 50%, the method comprising employing a catheter according to claim 1.

17. A method of reducing mechanical irritation of a ventricular wall during a medical diagnostic procedure, the method comprising employing a catheter having an operative head which responds to an applied contractile force with a low resistance to deliver contrast material for the procedure.

18. A method according to claim 17, wherein said catheter is sufficiently elastic to alternate between a first contracted conformation and a second extended conformation in accord with a degree of externally applied force.

19. An intraventricular catheter for delivery of contrast material for ventriculography, the catheter comprising:
(a) an operative distal head comprising a tubular body including at least two flexible turns; and
(b) a plurality of contrast material ports distributed along said tubular body between at least two of said at least two turns.

20. A catheter according to claim 19, wherein said operative distal head defines a transverse proximal aspect that is larger than a transverse distal aspect.

21. An intraventricular catheter for delivery of contrast material for ventriculography, the catheter comprising a pre-defined angle at an extraventricular portion of the catheter which directs a distal portion of the catheter to a correct position during use.
